(19) United States
(12) Patent Application Publication
(10) Pub. No.: US 2014/0113888 A1
(43) Pub. Date: Apr. 24, 2014

(54) Novel Combination of Therapeutic Agents

(71) Applicant: Glenn Crater, North Mississauga (CA)

(72) Inventor: Glenn Crater, North Mississauga (CA)

(73) Assignee: Glaxo Group Limited, Brentford, Middlesex (GB)

(21) Appl. No.: 14/124,276

(22) PCT Filed: Jun. 1, 2012
(86) PCT No.: PCT/EP2012/060444

§ 371 Date: Dec. 6, 2013

Related U.S. Application Data
(60) Provisional application No. 61/494,600, filed on Jun. 8, 2011.

(51) Int. Cl. A61K 31/58; A61K 9/00; A61K 31/439

(52) U.S. Cl. CPC A61K 31/58; A61K 31/439; A61K 9/0075
USPC 514/171

(57) ABSTRACT

The present invention is directed to the combination of a muscarinic receptor antagonist and a corticosteroid, and the use of said combination in treating diseases mediated via the M₃ muscarinic acetylcholine receptor and/or the glucocorticoid receptor, such as asthma.

### FIELD OF THE INVENTION

This invention relates to combinations of therapeutic agents for use in the treatment of diseases mediated via the M₃ muscarinic acetylcholine receptor and/or the glucocorticoid receptor, for example in the treatment of an inflammatory or respiratory tract disease, such as asthma.

More particularly, this invention relates to the combination of a muscarinic receptor antagonist and a corticosteroid, and the use of said combination in treating diseases mediated via the M₃ muscarinic acetylcholine receptor and/or the glucocorticoid receptor.

More particularly, this invention is concerned with novel pharmaceutical combination products comprising 4-[hydroxy(diphenyl)methyl]-1-{2-[(phenylmethyl)oxy]ethyl}-1-azoniabicyclo[2.2.2]octane bromide (umeclidinium bromide) and 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), and the use of said combination products in medicine, particularly in treating diseases mediated via the M₃ muscarinic acetylcholine receptor and/or the glucocorticoid receptor, for example in the treatment of an inflammatory or respiratory tract disease, such as asthma.

### BACKGROUND OF THE INVENTION

Asthma is a chronic inflammatory disorder of the airways, which affects approximately 300 million people worldwide. The disorder is characterised by widespread, variable and reversible airflow obstruction, and asthmatic patients typically experience episodes of breathlessness, chest tightness, wheezing and coughing.

The British Guidelines on the Management of Asthma (British Thoracic Society) state that the pharmacological management of asthma when regular preventer therapy is required may involve the use of glucocorticosteroids, beta-adrenoreceptor agonists, chromones, leukotriene receptor antagonists and theophyllines. Prescription of a corticosteroid is the current first choice therapy. For those asthmatic patients that are inadequately controlled on a low dose of a corticosteroid, an additional therapy can be introduced. The first choice add-on therapy to a corticosteroid is an inhaled long-acting beta-adrenoreceptor agonist (LABA). If symptom control is still inadequate following addition of a LABA to a patient's treatment plan, the dose of corticosteroid may be increased. Furthermore, if the addition of a LABA has had no added benefit this may be stopped, with the patient continuing solely with the higher dose of corticosteroid. If the disease remains poorly controlled after addition of a LABA therapy, it is recommended that a leukotriene receptor antagonist, a theophylline or slow-release b-agonist tablets be considered.

The guidelines state that for regular preventer therapy, the use of a short-acting anticholinergic is generally of no value. Furthermore, treatment plans do not mention the use of long-acting anticholinergic agents, such as antimuscarinic agents, and no long-acting antimuscarinics have been approved for the treatment of asthma.

Corticosteroid monotherapy and combination therapy with a LABA have become established methods for the maintenance treatment of asthma. However, there exists a need for alternative therapies for the effective pharmacological management of asthma, for example, for those patients that are currently poorly controlled on any approved and recommended treatment plan.

Furthermore, there exists a need for alternative therapies that are able to improve patient compliance and hence efficacy over current treatment options.

### SUMMARY OF THE INVENTION

The present invention provides a novel pharmaceutical combination product comprising:

a) the compound of formula (I):

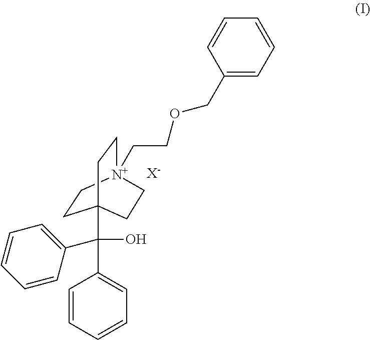

wherein X⁻ is a pharmaceutically acceptable anion;

and

b) a corticosteroid.

In one embodiment, the pharmaceutical combination product does not comprise any further therapeutically active agents.

In a further embodiment, the compound of formula (I) is umeclidinium bromide.

In a further embodiment, the corticosteroid is fluticasone furoate.

In a further embodiment, the pharmaceutical combination product comprises umeclidinium bromide and fluticasone furoate.

In a further embodiment, the present invention provides pharmaceutical combination products as herein disclosed for use in therapy, particularly in the treatment of inflammatory and respiratory tract diseases, such as asthma.

In a further embodiment, the present invention provides a method for the treatment of an inflammatory or respiratory disease, such as asthma, comprising administering to a patient in need thereof, a pharmaceutical combination product comprising the compound of formula (I) and a corticosteroid.

### DETAILED DESCRIPTION OF THE INVENTION

The present invention provides a pharmaceutical combination product comprising

a) the compound of formula (I):

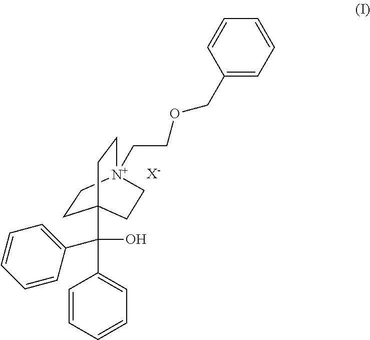

wherein X⁻ is a pharmaceutically acceptable anion;

and

b) a corticosteroid.

In one embodiment, the pharmaceutical combination product does not comprise any further therapeutically active agents.

As used herein, the term “therapeutically active agent” means any compound that is used to treat or prevent any disease or undesirable medical condition or a manifestation thereof, which afflicts a subject.

The pharmaceutically acceptable anion depicted by X⁻ may be selected from chloride, bromide, iodide, hydroxide, sulfate, nitrate, phosphate, acetate, trifluoroacetate, fumarate, citrate, tartrate, oxalate, succinate, mandelate, methanesulfonate or p-toluenesulfonate. In one embodiment the pharmaceutically acceptable anion X⁻ is bromide.

The structural formula for the quaternary moiety (free cation) of the compound of formula (I) is also referred to as 4-[hydroxy(diphenyl)methyl]-1-{2-[(phenylmethyl)oxy]ethyl}-1-azoniabicyclo[2.2.2]octane.

In a further embodiment of the invention, the compound of formula (I) is umeclidinium bromide.

The corticosteroid present in the pharmaceutical combination product may be in a free acid or base form, or as a pharmaceutically acceptable salt thereof.

In one embodiment, the corticosteroid is selected from the group consisting of fluticasone propionate, mometasone furoate, ciclesonide, budesonide and fluticasone furoate.

In one embodiment, the corticosteroid is fluticasone furoate.

In a further embodiment, the corticosteroid is fluticasone propionate.

In one embodiment, the pharmaceutical combination product of the invention comprises umeclidinium bromide and fluticasone furoate.

Umeclidinium bromide has been the subject of studies in animal models, and in humans, and has been found to be a long acting high-affinity pan-active muscarinic receptor antagonist which has potential for once-daily administration for the treatment of chronic obstructive pulmonary disease (COPD).

Fluticasone furoate is an inhaled corticosteroid that is still active 24 hours after dosing. In clinical studies it has been shown to be well-tolerated and has potential for once-daily administration for the maintenance treatment of asthma.

In a further embodiment, the pharmaceutical combination product of the invention comprises umeclidinium bromide and fluticasone propionate.

A pharmaceutical combination product comprising the compound of formula (I) and a corticosteroid may have use in the treatment of inflammatory or respiratory tract diseases, such as asthma.

Asthma is a chronic condition, which is characterised by widespread, variable and reversible airflow obstruction. Symptoms include coughing, wheezing, breathlessness and/or a tight feeling in the chest. Asthma attacks are generally caused by exposure to a trigger, such as pollen, dust or other allergens, which causes constriction of the airways (bronchoconstriction). It will be appreciated that a subject suffering from a condition such as asthma, may variously from time to time display no overt symptoms of the condition, or may suffer from periodic attacks during which symptoms are displayed or may experience exacerbations or worsening of the condition. In this context the term ‘treatment’ is intended to encompass prevention of such periodic attacks or exacerbations of the existing condition. Such treatment may be referred to as ‘maintenance treatment’ or ‘maintenance therapy’.

The amounts of the compound of formula (I) and the selected corticosteroid, and in one embodiment of the invention, umeclidinium bromide and fluticasone furoate, required to achieve a therapeutic effect will, of course, vary with the route of administration, the subject under treatment, the particular disorder or disease being treated, and the severity of the disease. In one embodiment, the route of administration is by inhalation via the mouth or nose. In a further embodiment, the route of administration is by inhalation via the mouth.

In one embodiment the compound of formula (I), and specifically umeclidinium bromide, may be administered by inhalation to deliver a dose of 4-[hydroxy(diphenyl)methyl]-1-{2-[(phenylmethyl)oxy]ethyl}-1-azoniabicyclo[2.2.2]octane (the free cation) of from about lmcg to about 1000 mcg/daily, e.g. 100, 250 or 500 mcg per day. In a further embodiment, the compound of formula (I) and specifically umeclidinium bromide may be administered by inhalation to deliver a dose of the free cation of 15.625 mcg, 31.25 mcg, 62.5 mcg or 125 mcg once or twice daily. In general, the compound of formula (I) will be administered once-daily.

In a further embodiment, the compound of formula (I), and specifically umeclidinium bromide, may be administered by inhalation, once daily, to deliver a dose of the free cation of 15.625 mcg per day.

In a further embodiment, the compound of formula (I), and specifically umeclidinium bromide, may be administered by inhalation, once daily, to deliver a dose of the free cation of 31.25 mcg per day.

In a further embodiment, the compound of formula (I), and specifically umeclidinium bromide, may be administered by inhalation, once daily, to deliver a dose of the free cation of 62.5 mcg per day.

In a further embodiment, the compound of formula (I), and specifically umeclidinium bromide, may be administered by inhalation, once daily, to deliver a dose of the free cation of 125 mcg per day.

The chosen corticosteroid may be administered, for example, by inhalation at a dose of from about 1 mcg to about 1000 mcg/day (calculated as the free base). When the corticosteroid is fluticasone furoate it may be administered by inhalation at a dose from about 25 mcg to about 800 mcg daily, and if necessary in divided doses. Thus, the daily dose of fluticasone furoate may be for example 25, 50, 100, 200, 300, 400, 600 or 800 mcg. In general, the dose of fluticasone furoate will be administered once-daily.

In one embodiment, the daily dose of fluticasone furoate is 200 mcg. In a further embodiment, the daily dose of fluticasone furoate is 100 mcg. In yet a further embodiment, the daily dose of fluticasone furoate is 50 mcg.

When the corticosteroid is fluticasone propionate it may be administered by inhalation at a dose from about 100 mcg to about 500 mcg daily, and if necessary in divided doses. Thus, the daily dose of fluticasone propionate may be for example 100, 250 or 500 mcg.

In a further embodiment, the present invention provides a pharmaceutical combination product for once-daily administration by inhalation, comprising umeclidinium bromide at a dose of the free cation of 125 mcg per day, and fluticasone furoate at a dose of 100 mcg per day.

In a further embodiment, the present invention provides a pharmaceutical combination product for once-daily administration by inhalation, comprising umeclidinium bromide at a dose of the free cation of 62.5 mcg per day, and fluticasone furoate at a dose of 100 mcg per day.

The individual compounds of the pharmaceutical combination product as described herein may be administered either sequentially or simultaneously. When administered sequentially the individual compounds may be presented in separate compositions or a combined composition. Furthermore, the compound of formula (I) and a corticosteroid may, be formulated separately and also presented in separate packs or devices for administration, or said individually formulated compounds may be presented in a single pack or device for administration. Where appropriate, the individual compounds may be admixed within the same composition, and presented therefore as a fixed pharmaceutical combination.

The compositions described above will in general include pharmaceutical carriers or excipients as described hereinafter, but combinations of the compounds without any excipients are also within the ambit of this invention.

In further aspects the invention therefore provides:

A pharmaceutical combination product comprising the compound of formula (I) and a corticosteroid presented separately for sequential or simultaneous administration;

A pharmaceutical combination product comprising the compound of formula (I) and a corticosteroid presented separately but held in the same pack or device, for sequential or simultaneous administration; and

A pharmaceutical combination product comprising the compound of formula (I) and a corticosteroid in admixture with each other for simultaneous administration.

In each case, each of the compound of formula (I) and/or a corticosteroid may be formulated with or without pharmaceutical carriers or excipients.

The present invention further provides a pharmaceutical combination product comprising the compound of formula (I) and a corticosteroid wherein at least one of said compounds is formulated with a pharmaceutically acceptable carrier or excipient.

The present invention further provides a pharmaceutical combination product comprising the compound of formula (I) and a corticosteroid wherein each compound is formulated with a pharmaceutically acceptable carrier or excipient.

In one embodiment of this invention, compositions of the compound of formula (I) and a corticosteroid include those suitable for inhalation, including fine particle powders, or mists which may be generated and administered by means of various types of inhalers for example, reservoir dry powder inhalers, unit-dose dry powder inhalers, pre-metered multi-dose dry powder inhalers, nasal inhalers or pressurized metered dose inhalers, nebulisers or insufflators.

The compositions may be prepared by any of the methods well known in the art of pharmacy. In general, said methods include the step of bringing the active ingredient(s) into association with the carrier which constitutes one or more accessory ingredients. In general the compositions are prepared by uniformly and intimately bringing into association the active ingredient with liquid carriers or finely divided solid carriers or both and then, if necessary, shaping the product into the desired composition.

Active ingredients for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced may be size reduced by conventional means e.g. by micronization. The desired fraction may be separated out by air classification or sieving. Preferably, the particles will be crystalline.

Powder compositions generally contain a powder mix for inhalation of the active ingredient and a suitable powder base (carrier/diluent/excipient substance) such as mono-, di or poly-saccharides (e.g. lactose or starch). Use of lactose is preferred. The lactose may be for example anhydrous lactose or α-lactose monohydrate. In one embodiment, the carrier is α-lactose monohydrate. Dry powder compositions may also include, in addition to the active ingredient and carrier, a further excipient (eg a ternary agent) such as a sugar ester, calcium stearate or magnesium stearate. Alternatively, the active ingredient may be presented without excipients. For the avoidance of doubt use of the term ‘composition’ or ‘formulation’ herein refers to the active ingredients either with or without excipients or carriers.

Dry powder compositions according to the invention may comprise a carrier. The carrier when it is lactose e.g. α-lactose monohydrate, may form from about 91 to about 99%, e.g. 97.7-99.0% or 91.0-99.2% by weight of the formulation. In general, the particle size of the carrier, for example lactose, will be much greater than the inhaled medicament within the present invention. When the carrier is lactose it will typically be present as milled lactose, having a MMD (mass median diameter) of 60-90 μm.

The lactose component may comprise a fine lactose fraction. The ‘fine’ lactose fraction is defined as the fraction of lactose having a particle size of less than 7 μm, such as less than 6 μm, for example less than 5 μm. The particle size of the ‘fine’ lactose fraction may be less than 4.5 μm. The fine lactose fraction, if present, may comprise 2 to 10% by weight of the total lactose component, such as 3 to 6% by weight fine lactose, for example 4.5% by weight fine lactose.

The present invention further provides a pharmaceutical combination product comprising the compound of formula (I) and a corticosteroid wherein at least one of these two compounds is formulated with a pharmaceutically acceptable carrier and a ternary agent.

The present invention further provides a pharmaceutical combination product comprising the compound of formula (I) and a corticosteroid wherein the compound of formula (I) is formulated with a pharmaceutically acceptable carrier and a ternary agent.

In one embodiment, said ternary agent is magnesium stearate.

Magnesium stearate, if present in the composition, is generally used in an amount of about 0.2 to 2%, e.g. 0.6 to 2% or 0.5 to 1.75%, e.g. 0.6%, 0.75%, 1%, 1.25% or 1.5% w/w, based on the total weight of the composition. The magnesium stearate will typically have a particle size in the range 1 to 50 μm, and more particularly 1-20 μm, e.g. 1-10 μm. Commercial sources of magnesium stearate include Peter Greven, Covidien/Mallinckodt and FACI.

In a further embodiment there is provided a pharmaceutical combination product comprising the compound of formula (I) and a corticosteroid wherein the compound of formula (I) is umeclidinium bromide and is presented as a dry powder composition containing magnesium stearate at an amount of 0.6% w/w based on the total weight of the composition.

The compositions may be presented in unit dosage form. Dry powder compositions for topical delivery to the lung by inhalation may, for example, be presented in capsules and cartridges of for example gelatine, or blisters of for example laminated aluminium foil, for use in an inhaler or insufflator.

Each capsule, cartridge or blister may generally contain between 1 mcg-1000 mcg, e.g. 100 to 500 mcg of the compound of formula (I) and/or between 1 mcg-1000 mcg, e.g. 1 to 200 mcg of a corticosteroid. Packaging of the formulation may be suitable for unit dose or multi-dose delivery.

As indicated above the compound of formula (I) and a corticosteroid may be formulated independently or in admixture. Said compounds may thus be incorporated in separate unit doses or may be combined in a single unit dose with or without additional carriers and/or excipients as deemed necessary.

In a further embodiment, each capsule, cartridge or blister may contain 15.625 mcg, 31.25 mcg, 62.5 mcg or 125 mcg of the free cation of the compound of formula (I).

In yet a further embodiment, each capsule, cartridge or blister may contain 15.625 mcg, 31.25 mcg, 62.5 mcg or 125 mcg of the free cation of umeclidinium bromide and/or 100 mcg or 200 mcg of fluticasone furoate.

In one embodiment, a composition suitable for inhaled administration may be incorporated into a plurality of sealed dose containers provided on medicament pack(s) mounted inside a suitable inhalation device. The containers may be rupturable, peelable or otherwise openable one-at-a-time and the doses of the dry powder composition administered by inhalation through a mouthpiece of the inhalation device, as known in the art. The medicament pack may take a number of different forms, for instance a disk-shape or an elongate strip. Representative inhalation devices are the DISKHALER™ and DISKUS™ devices, marketed by GlaxoSmithKline. The DISKUS™ inhalation device is, for example, described in GB 2242134A.

A dry powder inhalable composition may also be provided as a bulk reservoir in an inhalation device, the device then being provided with a metering mechanism for metering a dose of the composition from the reservoir to an inhalation channel where the metered dose is able to be inhaled by a patient inhaling at a mouthpiece of the device. Exemplary marketed devices of this type are TURBUHALER™ of AstraZeneca, TWISTHALER™ of Schering and CLICKHALER™ of Innovata.

A further delivery method for a dry powder inhalable composition is for metered doses of the composition to be provided in capsules (one dose per capsule) which are then loaded into an inhalation device, typically by the patient on demand. The device has means to rupture, pierce or otherwise open the capsule so that the dose is able to be entrained into the patient's lung when they inhale at the device mouthpiece. As marketed examples of such devices there may be mentioned ROTAHALER™ of GlaxoSmithKline and HANDIHALER™ of Boehringer Ingelheim.

A dry powder composition may also be presented in a delivery device which permits separate containment of the compound of formula (I) and a corticosteroid optionally in admixture with one or more excipients. Thus, for example, the individual compounds of the combination are administrable simultaneously but are stored separately, e.g. in separate pharmaceutical compositions, for example as described in WO 2003/061743 A1, WO 2007/012871 A1 and/or WO2007/068896. In one embodiment a delivery device permitting separate containment of actives is an inhaler device having two medicament packs in peelable blister strip form, each pack containing pre-metered doses in blister pockets arranged along its length. Said device has an internal indexing mechanism which, each time the device is actuated, peels opens a pocket of each strip and positions the packs so that each newly exposed dose of each pack is adjacent a manifold which communicates with a mouthpiece of the device. When the patient inhales at the mouthpiece, each dose is simultaneously drawn out of its associated pocket into the manifold and entrained via the mouthpiece into the patient's respiratory tract. Thus, each time the device is used, the patient is administered a combination therapy consisting of a dose from each medicament pack. A further device that permits separate containment of different compounds is DUGHALER™ of Innovata.

In a further embodiment, the present invention provides a dry powder inhaler (Inhaler 1) comprising two compositions presented separately, wherein the first composition comprises
i. umeclidinium bromide, and
ii. lactose, and
iii. magnesium stearate at an amount of about 0.6% w/w based on the total weight of the first composition;

and the second composition comprises
i. fluticasone furoate, and
ii. lactose.

In a further embodiment, the present invention provides Inhaler 1 wherein each composition is in unit dose form.

In a further embodiment, the present invention provides Inhaler 1 wherein the unit dose form is a capsule, cartridge or blister.

In a further embodiment, the present invention provides Inhaler 1 wherein umeclidinium bromide is present in an amount to deliver 125 mcg/dose of the free cation.

In a further embodiment, the present invention provides Inhaler 1 wherein umeclidinium bromide is present in an amount to deliver 62.5 mcg/dose of the free cation.

In a further embodiment, the present invention provides Inhaler 1 wherein umeclidinium bromide is present in an amount to deliver 31.25 mcg/dose of the free cation.

In a further embodiment, the present invention provides Inhaler 1 wherein umeclidinium bromide is present in an amount to deliver 15.625 mcg/dose of the free cation.

In a further embodiment, the present invention provides Inhaler 1 wherein fluticasone furoate is present in an amount of 100 mcg/dose.

In a further embodiment, the present invention provides Inhaler 1 wherein fluticasone furoate is present in an amount of 200 mcg/dose.

Spray compositions for inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the pharmaceutical product and a suitable propellant such as a fluorocarbon or hydrogen-containing chlorofluorocarbon or mixtures thereof, particularly hydrofluoroalkanes, especially 1,1,1,2-tetrafluoroethane, 1,1,1,2,3,3,3-heptafluoro-n-propane or a mixture thereof. The aerosol composition may optionally contain additional formulation excipients well known in the art such as surfactants e.g. oleic acid, lecithin or an oligolactic acid derivative e.g. as described in WO94/21229 and WO98/34596 and/or cosolvents e.g. ethanol. Pressurised formulations will generally be retained in a canister (e.g. an aluminium canister) closed with a valve (e.g. a metering valve) and fitted into an actuator provided with a mouthpiece.

There is thus provided as a further aspect of the invention a pharmaceutical combination product comprising the compound of formula (I) and a corticosteroid formulated individually or in admixture, with a fluorocarbon or hydrogen-containing chlorofluorocarbon as propellant, optionally in combination with a surface-active agent and/or a co-solvent. According to another aspect of the invention, the propellant is selected from 1,1,1,2-tetrafluoroethane, 1,1,1,2,3,3,3-heptafluoro-n-propane and mixtures thereof.

Another aspect of the invention is a pharmaceutical combination product consisting of the compound of formula (I) and a corticosteroid formulated individually or in admixture, with a fluorocarbon or hydrogen-containing chlorofluorocarbon as propellant, optionally in combination with a surface-active agent and/or a cosolvent. In another embodiment of the invention the propellant is selected from 1,1,1,2-tetrafluoroethane, or 1,1,1,2,3,3,3-heptafluoro-n-propane and mixtures thereof.

Where appropriate compositions according to the invention may be buffered by the addition of suitable buffering agents.

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulization may be formulated with an aqueous vehicle with the addition of agents such as acid or alkali, buffer salts, isotonicity adjusting agents or antimicrobials. They may be sterilized by filtration or heating in an autoclave, or presented as a non-sterile product.

Umeclidinium bromide, also referred to as 4-[hydroxy(diphenyl)methyl]-1-{2-[(phenylmethyl)oxy]ethyl}-1-azoniabicyclo[2.2.2]octane bromide, is described as Example 84, in WO2005/104745 (Glaxo Group Limited), which is incorporated by reference herein.

Fluticasone furoate, also referred to as 6α,9α-difluoro-17α-[(2-furanylcarbonyl)oxy]-11β-hydroxy-16α-methyl-3-oxo-androsta-1,4-diene-17β-carbothioic acid 5-fluoromethyl ester, is described as Example 1 in WO02/12265 (Glaxo Group Limited), which is incorporated by reference herein.

Clinical Studies

Umeclidinium Bromide

Umeclidinium bromide has been found to be an effective long-acting potent, pan-active antimuscarinic bronchodilator which demonstrates slow reversibility at the human M3 receptor in vitro and long duration of action in vivo when administered directly to the lungs in pre-clinical models. The long duration of action of this compound identified using in vitro models, when administered via inhalation in animals, and subsequently in early phase studies in healthy volunteers and COPD subjects supports the potential for use of this compound as a once daily bronchodilator for COPD.

Several clinical pharmacology studies have been conducted using umeclidinium bromide in both healthy volunteers and COPD patients to investigate the safety, tolerability, pharmacokinetics and pharmacodynamics of this compound. The bronchodilatory effects and duration of action of single inhaled doses of this compound as measured by plethysmography (sG_{aw}, R_{aw}) and spirometry (FEV₁) were assessed in some of the above noted studies. These studies showed clinically relevant bronchodilation and 24 h duration of action for the compound.

Throughout, when the dose of umeclidinium bromide is given, this relates to the active moiety 4-[hydroxy(diphenyl)methyl]-1-{2-[(phenylmethyl)oxy]ethyl}-1-azoniabicyclo[2.2.2]octane i.e the free cation rather than the salt.

In one such study, designed to evaluate the safety, efficacy and pharmacokinetics of umeclidinium bromide in subjects with COPD, five once-daily doses (62.5 mcg, 125 mcg, 250 mcg, 500 mcg and 1000 mcg), taken over a 14-day treatment period, produced statistically significant improvements in pulmonary function compared to placebo. All once-daily doses showed numerically greater improvement in trough FEV₁ than the open label tiotropium active control (18 mcg once-daily). In addition, this study confirmed that umeclidinium bromide has a once-daily profile.

A further study evaluated the efficacy and safety of three doses (125 mcg, 250 mcg and 500 mcg) of umeclidinium bromide administered once-daily via a dry powder inhaler over a 28 day period in subjects with COPD. This study confirmed that umeclidinium bromide appears to be safe and efficacious, maintaining significant bronchodilation over twenty four hours.

A further study in COPD patients shall evaluate the safety and efficacy of four doses (125 mcg, 62.5 mcg, 31.25 mcg and 15.625 mcg) administered once daily and two doses (31.25 mcg and 15.625 mcg) administered twice daily. Administration shall be via a dry powder inhaler.

Fluticasone Furoate

Several clinical pharmacology studies have been conducted using fluticasone furoate to investigate the safety and efficacy of this compound in asthmatic patients.

In one such study, the safety and efficacy of four doses of fluticasone furoate in subjects with persistent uncontrolled asthma were evaluated. In this study, which was a randomised, double-blind, placebo-controlled, parallel group study, 598 patients received one of six treatments: fluticasone furoate (25, 50, 100 or 200 mcg) once daily, fluticasone propionate 100 mcg twice daily or placebo for 8 weeks. The primary endpoint was change from baseline in trough (pre-dose) forced expiratory volume in 1 second (FEV₁) at Week 8. At Week 8, relative to placebo fluticasone furoate 50-200 mcg once daily had significantly greater increases in trough FEV₁ from baseline (p<0.05) with fluticasone furoate 100 mcg and 200 mcg achieving a >200 mL increase. This study supports the use of fluticasone furoate (100 or 200 mcg once-daily) for the treatment of persistant uncontrolled asthma.

Combination Therapy

A further study in patients with persistent asthma shall evaluate the dose-response of five doses of umeclidinium bromide (15.625 mcg, 31.25 mcg, 62.5 mcg, 125 mcg and 250 mcg) in combination with FF (100 mcg) administered once daily in the morning, and over a 14-day treatment period. As a secondary objective, the study shall compare the efficacy and safety of the five doses of umeclidinium bromide in combination with fluticasone furoate (100 mcg), with fluticasone furoate (100 mcg) alone or the fluticasone furoate/vilanterol trifenatate combination. Furthermore, the study will attempt to determine differential responses and its phenotypic characteristics by exploratory and subgroup analyses of umeclidinium bromide combined with fluticasone furoate, relative to fluticasone furoate alone and to the fluticasone furoate/vilanterol trifenatate combination.

Pharmaceutical Formulations

Preparation of Blends

Umeclidinium Bromide

Throughout, when the dose of umeclidinium bromide is given, this relates to the active moiety 4-[hydroxy(diphenyl)methyl]-1-{2-[(phenylmethyl)oxy]ethyl}-1-azoniabicyclo[2.2.2]octane i.e the free cation rather than the salt.

Pharmaceutical grade α-lactose monohydrate, sourced from DMV Fronterra Excipients, complying with the requirements of Ph.Eur/USNF may be used. Before use, the α-lactose monohydrate may be sieved through a coarse screen (for example with a mesh size 500 or 800 microns). The level of fines in the α-lactose monohydrate, which can be measured by Sympatec, may be 4.5% w/w less than 4.5 micron.

Umeclidinium bromide is micronised before use in an APTM microniser to give a mass median diameter of 1 to 5 microns, such as 2 to 5 microns.

Pharmaceutical grade magnesium stearate, sourced from Peter Greven, complying with the requirements of Ph.Eur/USNF may be used as supplied with a mass median particle size of 8 to 12 microns.

Blend A

Lactose monohydrate may be passed through a sieve and then combined with magnesium stearate and blended using either a high shear mixer (a QMM, PMA or TRV series mixer, such as TRV25 or TRV65) or a low shear tumbling blender (a Turbula mixer) to provide a magnesium stearate/lactose premix, hereinafter referred to as blend A.

Blend B

Final blend B may be obtained as follows. An quantity of blend A and compound (I) bromide may be screened, for example using a COMIL™, and then blended with the remaining blend A using either a high shear mixer (a QMM, PMA or TRV series mixer, such as TRV25 or TRV65) or a low shear tumbling blender (a Turbula mixer).

Representative Batch Formula for Umeclidinium Bromide Powder Blend (62.5 Microgram Per Blister)

| Ingredient | Quantity | |
|---|---|---|
| Micronised umeclidinium bromide | 74.1 | g |
| Magnesium Stearate | 75 | g |
| Lactose Monohydrate | To 12.5 | kg |

Note: 74.1 g is equivalent to 62.5 g of the free cation. The quantity of umeclidinium bromide added may be adjusted to reflect the assigned purity of the input drug substance.

Representative Batch Formula for Umeclidinium Bromide Powder Blend (125 Microgram Per Blister)

| Ingredient | Quantity | |
|---|---|---|
| Micronised umeclidinium bromide | 148.3 | g |
| Magnesium Stearate | 75 | g |
| Lactose Monohydrate | To 12.5 | kg |

Note: 148.3 g is equivalent to 125 g of the free cation. The quantity of Compound (I) Bromide added may be adjusted to reflect the assigned purity of the input drug substance.

Blending Parameters (Using a TRV25, 12.5 Kg Scale)

| Blend | Time (mins) | Approximate Speed (rpm) |
|---|---|---|
| A | 6 | 460 |
| B | 10 | 590 |

Blister Strip Preparation

The blended composition may then be transferred into blister strips (typical nominal mean quantity of blend per blister is 12.5-13.5 mg) of the type generally used for the supply of dry powder for inhalation and the blister strips were sealed in the customary fashion.

Powder blends of umeclidinium bromide for blisters containing other quantities of active, such as 31.25 mcg or 15.625 mcg per blister, may be prepared using the same procedure.

Fluticasone Furoate

Pharmaceutical grade α-lactose monohydrate, sourced from DMV Fronterra Excipients, complying with the requirements of Ph.Eur/USNF may be used. Before use, the α-lactose monohydrate may be sieved through a coarse screen (for example with a mesh size 500 or 800 microns). The level of fines in the α-lactose monohydrate, which can be measured by Laser Diffraction, may be 5 to 8% less than 4.5 micron.

fluticasone furoate is micronised before use in an APTM microniser to give a mass median diameter of 1 to 5 microns.

Blend

Lactose monohydrate may be passed through a sieve and then blended with fluticasone furoate using either a high shear mixer (a QMM, PMA or TRV series mixer, such as TRV25 or TRV65) or a low shear tumbling blender (a Turbula mixer).

Representative Batch Formula for Fluticasone Furoate

Powder Blend (100 Microgram Per Blister)

| Ingredient | Quantity | |
|---|---|---|
| fluticasone furoate | 84.0 | g |
| Lactose Monohydrate | To 10.5 | kg |

Representative Batch Formula for Fluticasone Furoate

Powder Blend (200 Microgram Per Blister)

| Ingredient | Quantity | |
|---|---|---|
| fluticasone furoate | 168.0 | g |
| Lactose Monohydrate | To 10.5 | kg |

Blending Parameters (Using a TRV25, 10.5 Kg Scale)

| | Time (mins) | Approximate Speed (rpm) |
|---|---|---|
| Blend | 7 | 550 |

Blister Strip Preparation

The blended composition may then be transferred into blister strips (typical nominal mean quantity of blend per blister is 12.5-13.5 mg) of the type generally used for the supply of dry powder for inhalation and the blister strips were sealed in the customary fashion.

Powder blends of fluticasone furoate for blisters containing other quantities of active, such as 25 mcg or 50 mcg per blister, may be prepared using the same procedure.

Example Dry Powder Inhaler Devices

Throughout, when the dose of umeclidinium bromide is given, this relates to the active moiety 4-[hydroxy(diphenyl)methyl]-1-{2-[(phenylmethyl)oxy]ethyl}-1-azoniabicyclo[2.2.2]octane i.e the free cation rather than the salt.

Umeclidinium bromide and fluticasone furoate may be administered by a dry powder inhaler containing two blister strips. One strip contains a blend of micronised umeclidinium bromide (15.625 mcg, 31.25 mcg, 62.5 mcg or 125 mcg per blister), magnesium stearate and lactose monohydrate. The second strip contains a blend of micronised fluticasone furoate (100 or 200 micrograms per blister), and lactose monohydrate. The DPI device will deliver, when actuated, the contents of a single blister simultaneously from each of the two blister strips.

Umeclidinium bromide and fluticasone furoate may be administered by a dry powder inhaler containing two blister strips. One strip contains a blend of micronised umeclidinium bromide (15.625 mcg, 31.25 mcg, 62.5 mcg or 125 mcg per blister), magnesium stearate (at an amount of 0.6% w/w of the total powder weight per blister) and lactose monohydrate. The second strip contains a blend of micronised fluticasone furoate (100 or 200 micrograms per blister), and lactose monohydrate. The DPI device will deliver, when actuated, the contents of a single blister simultaneously from each of the two blister strips.

Umeclidinium bromide and fluticasone furoate may be administered by a dry powder inhaler containing two blister strips. One strip contains a blend of micronised umeclidinium bromide (62.5 mcg or 125 mcg per blister), magnesium stearate (at an amount of 0.6% w/w of the total powder weight per blister) and lactose monohydrate. The second strip contains a blend of micronised fluticasone furoate (100 micrograms per blister), and lactose monohydrate. The DPI device will deliver, when actuated, the contents of a single blister simultaneously from each of the two blister strips.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

1. A pharmaceutical combination product comprising
a) a compound of formula (I): 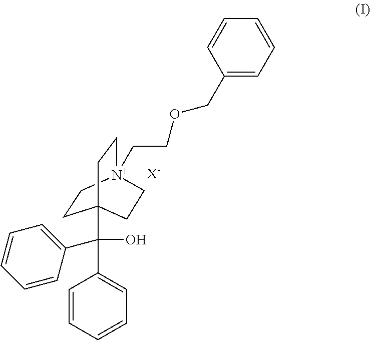
wherein X⁻ is a pharmaceutically acceptable anion; and
b) a corticosteroid.

2. A product according to claim 1, which does not comprise any further therapeutically active agents.

3. A product according to claim 1 or claim 2, wherein X⁻ is selected from the group consisting of chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate.

4. A product according to any of claims 1 to 3, wherein the compound of formula (I) is umeclidinium bromide.

5. A product according to any of claims 1 to 4, wherein the corticosteroid is selected from the group consisting of fluticasone propionate, mometasone furoate, ciclesonide, budesonide and fluticasone furoate.

6. A product according to claim 5, wherein the corticosteroid is fluticasone furoate.

7. A product according to any of claims 1 to 6, wherein the compound of formula (I) and the corticosteroid are presented in a form adapted for separate administration.

8. A product according to any of claims 1 to 6, wherein the compound of formula (I) and the corticosteroid are presented in a form adapted for sequential administration.

9. A product according to any of claims 1 to 6, wherein the compound of formula (I) and the corticosteroid are presented in a form adapted for simultaneous administration.

10. A product according to claim 9, wherein the compound of formula (I) and the corticosteroid are in admixture with each other.

11. A product according to any of claims 1 to 10, wherein at least one of the compound of formula (I) and the corticosteroid is formulated with a pharmaceutically acceptable carrier or excipient.

12. A product according to any of claims 1 to 11 in a form suitable for administration by oral or nasal inhalation.

13. A product according to claim 12, wherein the form is suitable for administration by inhalation via a medicament dispenser selected from a reservoir dry powder inhaler, a unit-dose dry powder inhaler, a pre-metered multi-dose dry powder inhaler, a nasal inhaler or a pressurized metered dose inhaler.

14. A product according to claim 13, wherein each of the compound of formula (I) and the corticosteroid is presented in the form of a dry powder composition.

15. A product according to claim 14, wherein the compound of formula (I) and the corticosteroid are presented as separate compositions.

16. A product according to claim 14, wherein the compound of formula (I) and the corticosteroid are presented as admixed compositions.

17. A product according to claim 15 or 16, wherein at least one of said compositions of the compound of formula (I) and the corticosteroid contains a carrier.

18. A product according to claim 15 or 16, wherein both compositions of the compound of formula (I) and the corticosteroid contain a carrier.

19. A product according to claim 17 or claim 18, wherein the carrier is lactose.

20. A product according to any of claims 14 to 19, wherein at least one of said compositions contains a ternary agent.

21. A product according to claim 20, wherein the ternary agent is magnesium stearate.

22. A product according to claim 21, wherein magnesium stearate is present in an amount of about 0.6% w/w in a composition of the compound of formula (I).

23. A product according to any of claims 14 to 22, wherein said separate or admixed compositions are in unit dose form.

24. A product according to claim 23, wherein the unit dose is in a capsule, cartridge or blister pack.

25. A product according to any of claims 14 to 24, wherein the composition is administered via a dry powder inhaler.

26. A product according to claim 25, wherein said inhaler permits separate containment of the compound of formula (I) and the corticosteroid.

27. A product according to claim 25 or 26, wherein the free cation of the compound of formula (I) is present in an amount of about 1 to 1000 mcg/dose.

28. A product according to claim 25 or 26, wherein the free cation of the compound of formula (I) is present in an amount of 125 mg/dose.

29. A product according to claim 25 or 26, wherein the free cation of the compound of formula (I) is present in an amount of 62.5 mg/dose.

30. A product according to claim 25 or 26, wherein the free cation of the compound of formula (I) is present in an amount of 31.25 mg/dose.

31. A product according to claim 25 or 26, wherein the free cation of the compound of formula (I) is present in an amount of 15.625 mcg/dose.

32. A product according to any of claim 25 or 31, wherein the corticosteroid is fluticasone furoate and is present in an amount of 100 mcg/dose.

33. A product according to any of claim 25 or 31, wherein the corticosteroid is fluticasone furoate and is present in an amount of 200 mcg/dose.

34. A dry powder inhaler containing a product as defined in any of claims 1 to 33.

35. A dry powder inhaler according to claim 34 comprising two compositions presented separately, wherein the first composition comprises
i. umeclidinium bromide, and
ii. lactose, and
iii. magnesium stearate at an amount of about 0.6% w/w based on the total weight of the first composition;
and the second composition comprises
i. fluticasone furoate, and
ii. lactose.

36. A dry powder inhaler according to claim 35, wherein each composition is in unit dose form.

37. A dry powder inhaler according to claim 36, wherein the unit dose form is a capsule, cartridge or blister.

38. A dry powder inhaler according to claim 36 or 37 wherein umeclidinium bromide is present in an amount to deliver 125 mcg/dose of the free cation.

39. A dry powder inhaler according to claim 36 or 37 wherein umeclidinium bromide is present in an amount to deliver 62.5 mcg/dose of the free cation.

40. A dry powder inhaler according to claim 36 or 37, wherein umeclidinium bromide is present in an amount to deliver 31.25 mcg/dose of the free cation.

41. A dry powder inhaler according to claim 36 or 37, wherein umeclidinium bromide is present in an amount to deliver 15.625 mcg/dose of the free cation.

42. A dry powder inhaler according to any of claims 36 to 41 wherein fluticasone furoate is present in an amount of 100 mcg/dose.

43. A dry powder inhaler according to any of claims 36 to 41 wherein fluticasone furoate is present in an amount of 200 mcg/dose.

44. A product according to claim 13, wherein each of the compound of formula (I) and the corticosteroid is presented in the form of a spray composition for inhalation.

45. A product according to claim 44, wherein the compound of formula (I) and the corticosteroid are presented as separate or admixed compositions.

46. A product according to claim 44 or 45, wherein the spray composition is an aqueous solution or suspension.

47. A product according to claim 44 or 45, wherein the spray composition is an aerosol composition.

48. A product according to claim 47, wherein the aerosol composition comprises a fluorocarbon or hydrogen-containing chlorofluorocarbon as propellant.

49. A product according to claim 48, wherein the propellant is a hydrofluoroalkane.

50. A product according to claim 49, wherein the propellant is 1,1,1,2-tetrafluoroethane, 1,1,1,2,3,3,3-heptafluoro-n-propane or a mixture thereof.

51. A product according to any of claims 47 to 50, further comprising a co-solvent.

52. A product according to any of claims 47 to 51, further comprising a surface-active agent.

53. A pressurised metered dose inhaler containing a product as defined in any of claims 1 to 13 and 47 to 52.

54. A product as defined in any of claims 1 to 53 for use in therapy.

55. A product as defined in any of claims 1 to 53 for use in the treatment of inflammatory or respiratory tract diseases.

56. A product for use according to claim 55, wherein the inflammatory or respiratory tract disease is asthma or COPD.

57. A product for use according to claim 56, wherein the product is administered once daily.

58. A method for the treatment of an inflammatory or respiratory tract disease, comprising administering to a patient in need thereof, a product as defined in any of claims 1 to 53.

59. A method of treatment according to claim 58, wherein the disease is asthma or COPD.

60. A method of treatment according to claim 59, wherein administration to a patient in need thereof is once-daily.
